(19) United States
(12) Patent Application Publication
(10) Pub. No.: US 2008/0177360 A1
(43) Pub. Date: Jul. 24, 2008

(54) Patient warming system

(76) Inventor: Kenneth L. Willeford, Shallotte, NC (US)

Correspondence Address: HODES, PESSIN & KATZ , P.A, 901 DULANEY VALLY ROAD , SUITE 400, BALTIMORE, MD, 21204, US

(21) Appl. No.: 11/657,171

(22) Filed: Jan. 24, 2007

(51) Int. Cl. A61F 7/12; A61M 1/14

(52) U.S. Cl. 607/105; 604/28

(57) ABSTRACT

A method of warming a patient during a surgical procedure under operating room conditions. The patient's bladder is catheterized to allow simultaneous filling and draining of the bladder. A pump and a fluid warmer are provided. The warmed fluid and urine are drained out of the bladder under passive gravity-flow conditions.

### CROSS REFERENCE TO RELATED APPLICATION

This application is related to U.S. application Ser. No. 60/762,240 filed Jan. 26, 2006.

### BACKGROUND OF THE INVENTION

1. Field of the Invention

The present invention relates to prevention of patient hypothermia during a surgical procedure.

2. Description of Related Art

Intraoperative hypothermia of surgical patients is a common problem with well documented adverse events. Unintentional intraoperative heat loss occurs as a result of low ambient temperatures in the operating room, open exposed wounds, administration of cool intravenous fluids, cool irrigating fluids, sterile preparation of the surgical site with fluids creating evaporative losses, and the effect of anesthetic agents that impair the body's ability to thermoregulate by the hypothalamus with concurrent vasodilation increasing heat loss and reduced metabolism with decreased heat production. (Patients have their skin exposed, washed with cold soap in a cold room and given medications that prevent the normal thermoregulatory mechanisms).

Physiologic effects of hypothermia include decreased oxygen tension in the blood, decreased metabolism, decreased drug biotransformation, impaired renal transport processes, altered membrane excitability, changes in cardiac rate and rhythm, central nervous system depression, hyperglycemia, and sympathetic nervous system stimulation.

Outcome studies have confirmed an increase in wound infection, myocardial infarction, need for post operative mechanical ventilation, probability for blood transfusion, and mortality in hypothermic versus normothermic patients.

Several methods are currently employed to prevent hypothermia in surgical patients. These include insulation of the patient, humidification of inspired respiratory gases, warming of intravenous fluids, and forced air warming blankets.

In the prior art of which I am aware, Lasheras et al U.S. Pat. No. 6,648,906 disclosed an apparatus and a method for regulating patient temperature by irrigating the patient's bladder with a fluid. A catheter is inserted through the urethra and into the bladder, the catheter having at least two lumens; and a heated (or chilled) fluid is passed through a supply lumen of the catheter and into the bladder. The fluid is evacuated from the bladder through a return lumen of the catheter, and the quantity of urine flowing out of the bladder is monitored. The pressure of the fluid flowing into the supply lumen of the catheter, as well as to the return lumen, is also monitored.

As stated by Lasheras et al,—
Patients may require pre or post-operative cooling for a variety of reasons, including, for example, treatment of a malignant hypothermia crisis and induction of therapeutic hypothermia for neurosurgery.”
   —whereas my invention is intended to prevent hypothermia during a surgical procedure in the O.R.

### BRIEF SUMMARY OF THE INVENTION

In its broadest aspect, the present invention provides a unique method of warming a patient to prevent hypothermia in the patient during a surgical procedure under operating room conditions.

My improved method includes the steps of: 1) catheterization of the patient's bladder to allow for simultaneous filling and draining of the bladder; 2) providing a pump and a fluid warmer; and 3) draining the warmed fluid and urine out of the bladder under passive gravity-flow conditions to minimize potential complications of bladder rupture and/or thermal injury thereto.

Under gravity flow conditions, the pressure of the fluid flowing through the return lumen ceases to be a potential problem, and this is a unique feature and important advantage of my invention.

These and other objects of the present invention will become apparent from a reading of the following specification taken in conjunction with the enclosed drawings.

### BRIEF DESCRIPTION OF THE DRAWINGS

FIG. 1 is a diagram of the steps of the method of the present invention.

FIGS. 2-5 are photographs of a simulation of the system showing a balloon to represent the patient's bladder with a catheter, a urinary drainage bag, a fluid management system tubing set and a blood/fluid warming system tubing set.

### DETAILED DESCRIPTION OF THE INVENTION

The current invention utilizes continuous bladder irrigation with warm fluids to prevent hypothermia. This technique can affect patient temperature through two mechanisms. First, the bladder will become warm and dissipate this energy to adjacent tissues. More significantly, the arterial blood that supplies the bladder will leave through the veins at an elevated temperature and distribute this warmth systematically by the cardiovascular system. This mechanism can be significant as the bladder is quite a vascular organ with 6 supplying arteries and drained by a venous plexus. Tissue blood flow of the bladder has been measured as high as 74 milliliters per minute per one hundred grams of tissue.

Catheterization of the bladder is a routine and common procedure in surgical patients, particularly in longer operations where temperature regulation is a greater problem. The current prototype uses existing components and combines them in a unique manner to accomplish this task. It can be employed in any situation where the bladder is catheterized.

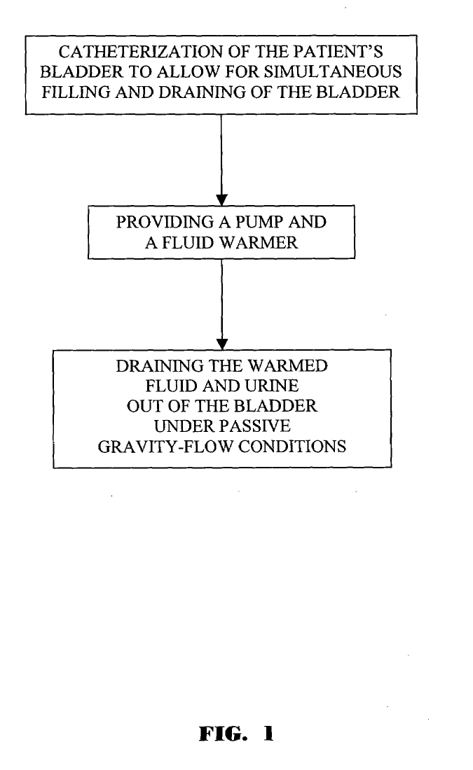

The method of the present invention is shown diagrammatically in FIG. 1.

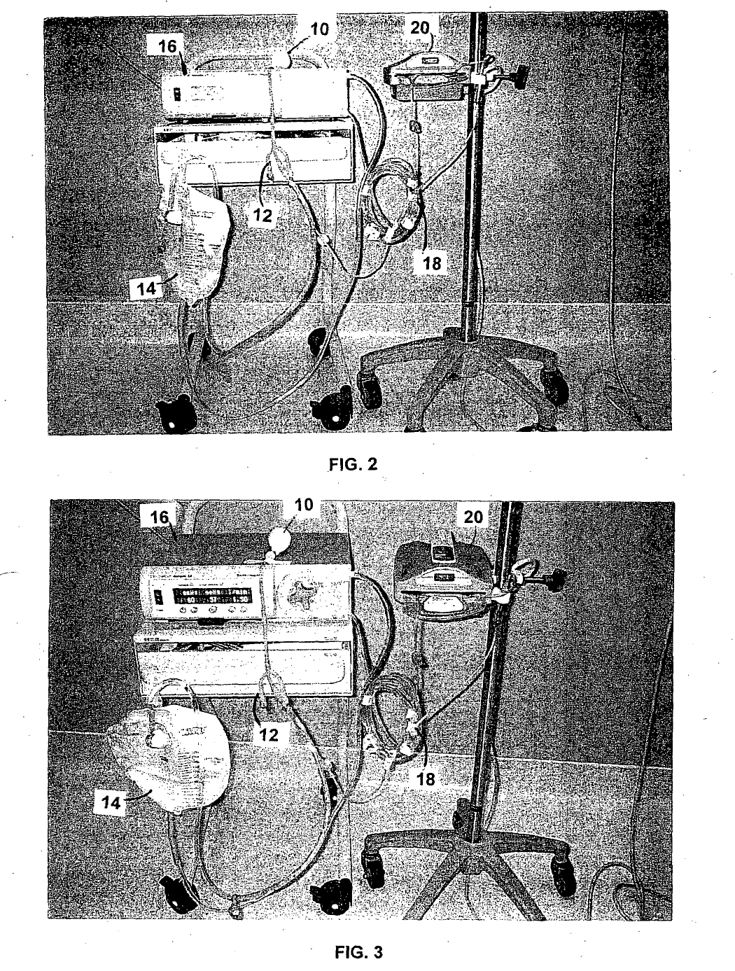

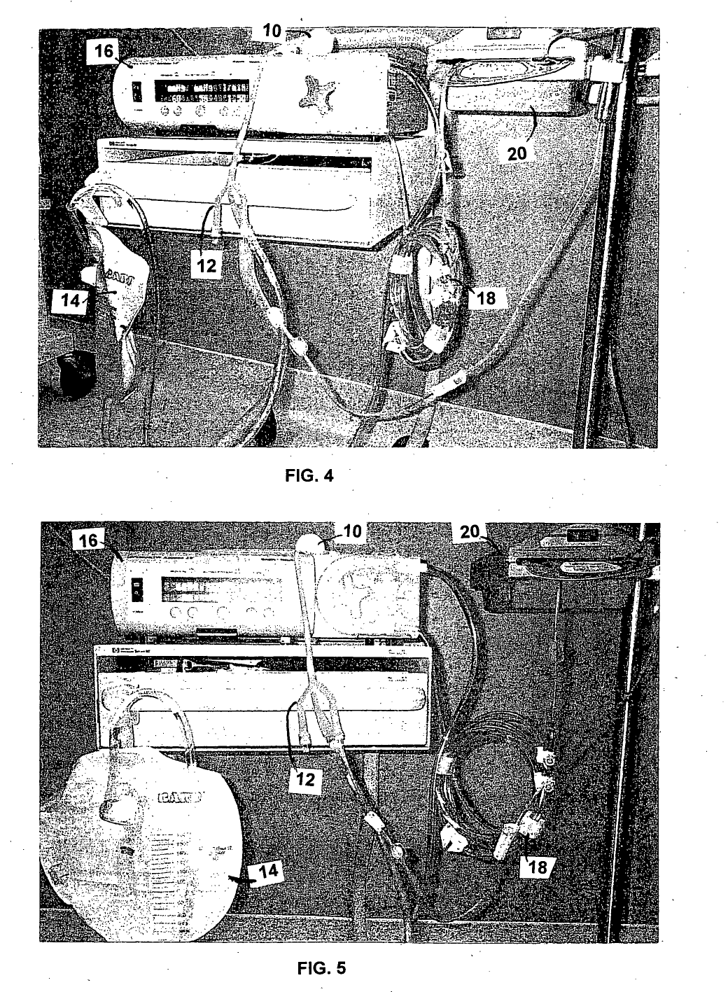

In FIGS. 2-5, the balloon 10 represents the bladder. A Bard Urological Lubricath® 18 french foley catheter 12 with a 5 ml balloon (#0119L18) is inserted into the bladder. The bladder is drained by gravity into a Bard urological® urinary drainage bag 14 (#154002). The drain tube of the urinary bag is connected to a Smith & Dyonics Access 15® (#7205699) fluid management system tubing set 16. The fluid and urine is then pumped through a Medex (#MX4312L) 3-way stopcock 18 and sequentially through a Ranger(® blood/fluid warming system tubing set 20 (#24200). The fluid continues through this system into the bladder via the 3-way foley.

The essential components include a 3-way foley to allow simultaneous filling and draining of the bladder 10, a pump, and a fluid warmer 20.

The Smith & Nephew Dyonics Access 15® (#7205699) fluid management system 16 is intended for use in orthopedic arthroscopy. It is a fluid pump where the flow rate and pressure can be adjusted. In this design with passive gravity drainage of the bladder there is no excess pressure in the bladder which would predispose the patient to the complication of bladder rupture. The prototype has been successfully bench tested with a pressure of 60 millimeters of mercury and a flow rate of 1.5 liters per minute.

The Ranger® blood/fluid warming system 20 is used for warming intravenous fluids and blood transfusions. It is preset so the effluent temperature is 41° Celsius. This is safe for intravenous fluids and would prevent complications of thermal injury. (The bladder can likely tolerate higher temperatures safely which would improve the effectiveness of this design for patient warming purposes; however the threshold temperature of the bladder lining to prevent injury has not been determined.)

The potential complications of bladder rupture and thermal injury have been addressed. There is an additional risk of infection, as with all procedures where the bladder is catheterized. All of the tubing components of this system are individually packaged in a sterile fashion and will need to be assembled with attention to sterile technique.

An additional consideration is the measurement of urine output. This is typically measured as the total fluid collected in the bag. This patient warming system will need to be primed with sterile fluid (saline), and the urine output measured as the increase in total fluid within the system.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. The method of warming a patient to prevent hypothermia in the patient during a surgical procedure under operating room conditions, comprising the steps of catheterization of the patient's bladder to allow for simultaneous filling and draining of the bladder, providing a pump and a fluid warmer, and draining the warmed fluid and urine out of the bladder under passive gravity-flow conditions to minimize potential complications of bladder rupture and/or thermal injury thereto.
